(19) United States
(12) Patent Application Publication
(10) Pub. No.: US 2024/0325369 A1
(43) Pub. Date: Oct. 3, 2024

(54) TREATMENT OF METASTATIC CASTRATION-RESISTANT PROSTATE CANCER WITH NIRAPARIB

(71) Applicant: Janssen Pharmaceutica NV, Beerse (BE)

(72) Inventors: Peter Francis, Raritan, NJ (US); Angela Mennicke LOPEZ-GITLITZ, Los Angeles, CA (US); Natalie A NUTNICK, Norristown, PA (US); Gary Edward MASON, New Hope, PA (US); Xin ZHAO, Foster City, CA (US); Michael P GORMLEY, Mount Laurel, NJ (US); Yuangen ZHU, Towaco, NJ (US); Karen Ann URTISHAK, Media, PA (US); Shibu THOMAS, Lower Gwynedd, PA (US); Adam A Del Corral, River Edge, NJ (US)

(21) Appl. No.: 18/580,583

(22) PCT Filed: Jul. 18, 2022
(86) PCT No.: PCT/EP2022/070026

§ 371 Date: Jan. 18, 2024

Related U.S. Application Data
(60) Provisional application No. 63/223,352, filed on Jul. 19, 2021.

(30) Foreign Application Priority Data
Aug. 11, 2021 (EP) 21190762.1

(51) Int. Cl. A61K 31/454; A61K 9/00; A61P 35/00

(52) U.S. Cl. CPC A61K 31/454; A61K 9/0053; A61P 35/00

(57) ABSTRACT

The present invention relates to a method of improving the efficacy of treatment of line 2+ metastatic castration-resistant prostate cancer (mCRPC) with biallelic DNA-repair anomalies in a male human, wherein said biallelic DNA-repair anomalies are selected from: i) BRCA (BRCA1, BRCA2, or a combination thereof), ii) non-BRCA (ATM, FANCA, PALB2, CHEK2, BRIP1, HDAC2, or any combination thereof): or iii) any combination thereof; wherein the male human has received prior taxane-based chemotherapy and androgen receptor (AR)-targeted therapy; said method of improving the efficacy of treatment comprising administering to said male human a once-daily oral dosing of 300 mg niraparib.

### TECHNICAL FIELD OF THE INVENTION

The present disclosure relates to methods of improving the treatment efficacy in patients having metastatic castration-resistant prostate cancer.

### BACKGROUND OF THE INVENTION

Prostate cancer is the most common non-cutaneous malignancy in men and the second leading cause of death in men from cancer in the western world.

Prostate cancer results from the uncontrolled growth of abnormal cells in the prostate gland. Once a prostate cancer tumor develops, androgens such as testosterone promote prostate cancer growth. At its early stages, localized prostate cancer is often curable with local therapy including, for example, surgical removal of the prostate gland and radiotherapy. However, when local therapy fails to cure prostate cancer, as it does in at least a third of men, the disease progresses into incurable metastatic disease (i.e., disease in which the cancer has spread from one part of the body to other parts).

Current therapeutic options for men with metastatic castration-resistant prostate cancer (mCRPC) that improve survival and limit progression include taxane-based chemotherapy, and androgen receptor-targeted agents such as abiraterone acetate plus prednisone, enzalutamide, or radium-223.

Platinum-based chemotherapy has been tested in a number of clinical studies in molecularly unselected prostate cancer patients with limited results and significant toxicities.

Niraparib is an orally available, highly selective poly(adenosine diphosphate [ADP]-ribose) polymerase (PARP) inhibitor, with activity against PARP-1 and PARP-2 deoxyribonucleic acid (DNA)-repair polymerases.

PARPs are enzymes responsible for repair of DNA single-strand breaks (SSBs) through a process called base excision repair. PARP inhibition leads to an accumulation of unrepaired SSBs, which result in stalling and collapse of replication forks and, consequently, to double-stranded breaks (DSBs). Normally, DSBs are repaired through homologous recombination (HR). If not repaired. DSBs result in cell death. When tumor cells with DNA-repair defects involving the HR pathway (e.g., Breast Cancer genes [BRCA]-1/2) are treated with a PARP inhibitor, they are unable to efficiently and accurately repair DSBs, which creates a synthetic lethal condition. In men with metastatic castration-resistant prostate cancers (mCRPC), tumors with DNA-repair anomalies account for approximately 20% to 30% of the sporadic cancers.

There is a need for improved methods of treatment of prostate cancer in patients who either do not respond initially or become refractory to the existing treatments.

### SUMMARY OF THE INVENTION

An objective of the present invention is to improve the efficacy of treatment of line 2+ mCRPC with biallelic DNA-repair anomalies selected from: i) BRCA (BRCA1, BRCA2, or a combination thereof), ii)non-BRCA (ATM, FANCA, PALB2, CHEK2, BRIP1, HDAC2, or any combination thereof), or iii) any combination thereof, in male humans having received prior taxane-based chemotherapy and AR-targeted therapy.

An objective of the present invention is to improve the efficacy of treatment of mCRPC in patients having more advanced stage of disease or heavy disease burden such as visceral disease (clinical manifestation of mCRPC with predominant lung and liver involvement), which carries a particularly ominous prognosis.

An objective of the present invention is to improve the efficacy of treatment of mCRPC in patients who have been heavily pre-treated and who have none or few other effective treatment options available to them, for example in patients in their third, fourth or fifth line of therapy or beyond.

An objective of the present invention is to provide an improved treatment of mCRPC that results in a stable to improved Health Related Quality of Life (HRQoL) in patients, which would be an extraordinary accomplishment in a difficult-to-treat population.

The present invention relates to a method of improving the efficacy of treatment of line 2+ metastatic castration-resistant prostate cancer (mCRPC) with biallelic DNA-repair anomalies in a male human, wherein said biallelic DNA-repair anomalies are selected from: i) BRCA (BRCA1, BRCA2, or a combination thereof), ii) non-BRCA (ATM, FANCA, PALB2, CHEK2, BRIP1, HDAC2, or any combination thereof); or iii) any combination thereof; wherein the male human has received prior taxane-based chemotherapy and androgen receptor (AR)-targeted therapy, said method of improving the efficacy of treatment comprising administering to said male human a once-daily oral dosing of 300 mg niraparib.

The improved efficacy is a median overall survival (OS) of about 12 months with a 95% confidence interval (95% CI).

In an embodiment, the male human has BRCA DNA-repair anomalies and the median OS is about 13 months (95% CI).

In an embodiment, the male human has non-BRCA DNA-repair anomalies and the median OS is about 10 months (95% CI).

The improved efficacy is also a median radiographic progression-free survival (rPFS) of about 5.6 months (95% CI).

In an embodiment, the male human has BRCA DNA-repair anomalies and the rPFS is about 8.1 months (95% CI).

In an embodiment, the male human has BRCA DNA-repair anomalies and the improved efficacy is an objective response rate (ORR) of about 34.2% (95% CI).

The improved efficacy is also a median duration of objective response of about 5.55 months (95% CI).

The improved efficacy is also a time to radiographic progression of about 5.8 months (95% CI). In an embodiment, the male human has BRCA DNA-repair anomalies and the time to radiographic progression is about 8.08 months (95% CI).

The improved efficacy is also a median time to PSA progression of about 4.6 months (95% CI).

The improved efficacy is also a median time to symptomatic skeletal event of about 13 months (95% CI).

The improved efficacy is also a circulating tumor cells (CTC) response rate of about 18% (95% CI).

In an embodiment, the male human has BRCA DNA-repair anomalies and the CTC response rate is about 24% (95% CI).

In an embodiment, the male human has non-BRCA DNA-repair anomalies and the CTC response rate is about 9% (95% CI).

In any one of the methods presented herein, the taxane-based chemotherapy is docetaxel, paclitaxel, or cabazitaxel.

In any one of the methods presented herein, the AR-targeted therapy encompasses i) surgical castration (orchiectomy); and/or ii) medical castration selected from the group consisting of, luteinizing hormone-releasing hormone (LHRH) agonists such as leuprorelin or leuprolide, goserelin, triptorelin, histrelin, nafarelin, gonadorelin, buserelin, and the like; LHRH antagonists such as degarelix, relugolix, and the like; abiraterone acetate (Zytiga®): ketoconazole: anti-androgens such as flutamide, nilutamide, bicalutamide, enzalutamide, apalutamide, darolutamide, and the like, and other androgen-suppressing drugs such as estrogens, diethylstilbestrol, and the like.

In any one of the methods presented herein, niraparib is in the salt form of tosylate monohydrate, sulfate, benzenesulfate, fumarate, succinate, camphorate, mandelate, camsylate, lauryl sulfate, or a mixture of tosylate monohydrate and lauryl sulfate.

The present invention relates as well to a once-daily oral dosing of 300 mg niraparib for use in any one of the methods, as presented herein, of improving the efficacy of treatment of line 2+ metastatic castration-resistant prostate cancer (mCRPC) with biallelic DNA-repair anomalies in a male human, wherein said biallelic DNA-repair anomalies are selected from: i) BRCA (BRCA1, BRCA2, or a combination thereof), ii) non-BRCA (ATM, FANCA, PALB2, CHEK2, BRIP1, HDAC2, or any combination thereof): or iii) any combination thereof; wherein the male human has received prior taxane-based chemotherapy and androgen receptor (AR)-targeted therapy.

### BRIEF DESCRIPTION OF THE DRAWINGS

FIG. 1 illustrates a Kaplan-Meier Plot of Overall Survival; ITT Analysis Set. ITT: BRCA or non BRCA subjects; BRCA: biallelic BRCA1 or BRCA2 or germline BRCA; non-BRCA; biallelic in ATM, FANCA, PALB2, CHEK2, BRIP1 or HDAC2.

FIG. 2 illustrates a Kaplan-Meier Plot of rPFS; ITT Analysis Set. ITT: BRCA or non BRCA subjects: BRCA: biallelic BRCA1 or BRCA2 or germline BRCA: non-BRCA; biallelic in ATM, FANCA, PALB2, CHEK2, BRIP1 or HDAC2.

FIG. 3 illustrates a Kaplan-Meier Plot of Time to Radiographic Progression; ITT Analysis Set. ITT: BRCA or non BRCA subjects; BRCA: biallelic BRCA1 or BRCA2 or germline BRCA; non-BRCA; biallelic in ATM, FANCA, PALB2, CHEK2, BRIP1 or HDAC2.

FIG. 4 illustrates a Waterfall Plot of Maximum Change in PSA from Baseline Any Time on the Study; ITT Analysis Set. The reference line represents 50% decrease. Increase or decrease greater than 100% is set to 100%. B=BRCA; N=NON-BRCA. ITT: BRCA or non BRCA subjects; BRCA: biallelic BRCA1 or BRCA2 or germline BRCA; non-BRCA: biallelic in ATM, FANCA, PALB2, CHEK2, BRIP1 or HDAC2.

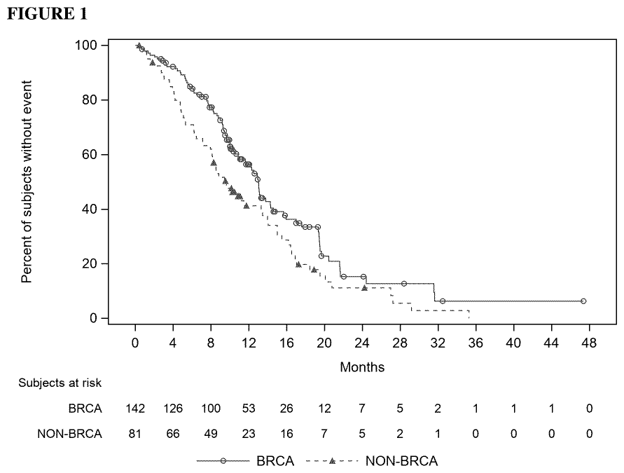

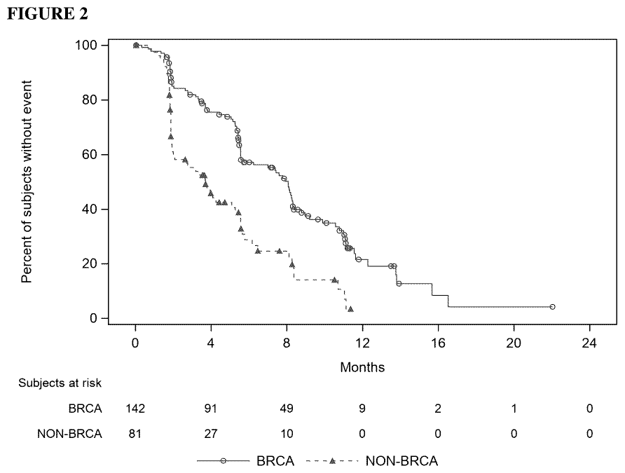

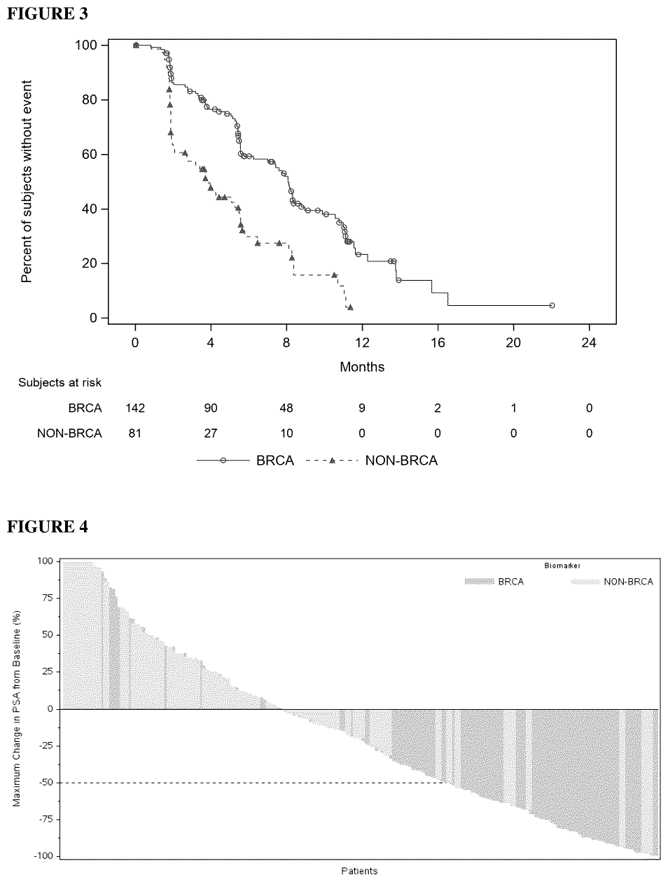

### DETAILED DESCRIPTION OF THE INVENTION

The present inventions may be understood more readily by reference to the following detailed description. It is to be understood that these inventions are not limited to the specific products, methods, conditions or parameters described and/or shown herein, and that the terminology used herein is for the purpose of describing particular embodiments by way of example only and is not intended to be limiting of the claimed inventions.

The entire disclosures of each patent, patent application, and publication cited or described in this document are hereby incorporated herein by reference.

As employed above and throughout the disclosure, the following terms and abbreviations, unless otherwise indicated, shall be understood to have the following meanings.

In the present disclosure the singular forms “a”, “an,” and “the” include the plural reference, and reference to a given numerical value includes at least that value, unless the context clearly indicates otherwise. Thus, for example, a reference to “an ingredient” is a reference to one or more of such ingredients and equivalents thereof known to those skilled in the art, and so forth. Furthermore, when indicating that a certain element “may be” X, Y. or Z, it is not intended by such usage to exclude in all instances other choices for the element.

When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” refers to a value of 7.2 to 8.8, inclusive; as another example, the phrase “about 8%” refers to a value of 7.2% to 8.8%, inclusive.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.”

### LIST OF ABBREVIATIONS OF TERMS

^{99m}Tc technetium-99m
AA-P abiraterone acetate plus prednisone
AE adverse event
ALT alanine aminotransferase
AML acute myeloid leukemia
ANC absolute neutrophil count
AR androgen receptor
AST aspartate aminotransferase
ATM Ataxia Telangiectasia Mutated gene
AUC area under the curve
BPI(-SF) Brief Pain Inventory (-Short Form) questionnaire
BRCA1 Breast Cancer gene 1
BRCA2 Breast Cancer gene 2
BRIP1 BRCA1 Interacting Protein C-terminal Helicase 1 gene
CBC complete blood count
CD4 cluster of differentiation glycoprotein
CDKN2A Cyclin Dependent Kinase inhibitor 2A
CHEK2 Checkpoint Kinase 2 gene
CHMP Committee for Medicinal Products for Human Use
CI confidence interval
CL/F Clearance
Cmax maximum concentration
COVID-19 Coronavirus Disease 2019
CR complete response
CSR clinical study report
CT computed tomography
CTC circulating tumor cells
Ctrough trough concentration
ctDNA circulating tumor DNA
CYP cytochrome P450
DNA deoxyribonucleic acid
DRD DNA-repair defects
DRC Data Review Committee
DSB double-stranded breaks
ECG Electrocardiogram
ECOG PS Eastern Cooperative Oncology Group Performance Status
eCRF electronic case report form
EMA European Medicines Agency
EoT End-of-Treatment
EQ-5D-5L Euro-QoL questionnaire
FACT-P Functional Assessment of Cancer Therapy-Prostate questionnaire
FANCA Fanconi Anemia Complementation Group A gene
FOIA Freedom of Information Act
GCP good clinical practice
GnRHa gonadotropin releasing hormone analog
HDAC2 Histone Deacetylase 2 gene
HIV human immunodeficiency virus
ICF informed consent form
ICH International Conference on Harmonisation
IEC Independent Ethics Committee
IRB Institutional Review Board
ITT intent-to-treat
M1 metabolite 1
mCRPC metastatic castration-resistant prostate cancer
MDS myelodysplastic syndrome
MedDRA Medical Dictionarv for Regulatory Activities
MRI magnetic resonance imaging
NCI-CTCAE National Cancer Institute—Common Terminology Criteria for Adverse Events
NGS next-generation sequencing
ORR objective response rate
OS overall survival
PALB2 partner and localizer of BRCA2 gene
PARP poly (adenosine diphosphate [ADP]-ribose) polymerase
PCWG3 Prostate Cancer Working Group 3
PK pharmacokinetic(s)
PR partial response
PRO patient-reported outcome(s)
PSA prostate-specific antigen
QT/QTc Q-T intenal/Q-T interval corrected
QTcF Q-T interval corrected using Fridericia's formula
RECIST Response Evaluation Criteria in Solid Tumors
RNA ribonucleic acid
rPFS radiographic progression-free survival
SAE serious adverse event
SAP statistical analysis plan
SOC system organ class
SSB single stranded break
SSE symptomatic skeletal event
TEAE treatment-emergent adverse event
Tmax time to maximum concentration
TMF trial master file
TP53 Tumor Protein 53
ULN upper limit of normal
VAS visual analogue scale
V/F volume of distribution
WHO World Health Organization

“Treat,” “treating” and “treatment” refers to an intervention aimed at the amelioration of the cancer by the inhibition, delay, delay of the rate, or halt of the progress of the cancer. Unless otherwise specified, the terms “treat” and “treatment” refers to the totality of effects described, but on other embodiments, the terms may also refer to any one of the effects described, or exclusive of at least one effect.

“Improving the efficacy” means achieving a better measure in one or more of the clinical outcomes or endpoints related to efficacy, when compared to the same measure when treating with Standard of Care (SOC) or other therapies available in the art, for the same or comparable cohort of patients and clinical stage of disease.

“Therapeutically effective amount” or “effective amount” means an amount of the therapeutic agent effective for treating a prostate cancer.

“Safe therapeutic” means an amount of the therapeutic agent that is safe for treating a prostate cancer.

“Once-daily” means a dosing regimen in which two or more dosage forms are administered once a day (q.d.), i.e., within a small period of time in a day. For example, the two, three, or four niraparib dosage forms, when administered once-daily, they are administered at the same time, or within one minute, two minutes, five minutes, 1 hour, or a maximum period of time that does not impair the pharmacokinetics and pharmacodynamics of niraparib achieved with the clinical study subject of the present invention. “Once-daily” is used in contrast to “multiple-daily”, the latter referring to two or more dosage forms being administered two times a day (bid), three times a day (tid), four times a day (qid), and so on.

The term “pharmaceutically acceptable” means that which is generally safe, non-toxic and neither biologically nor otherwise undesirable and includes that which are acceptable for human pharmaceutical use as well as veterinary use.

The term “androgen receptor” includes the wild-type androgen receptor as well as androgen-resistant ARs and/or AR mutants associated with castration-resistant prostate cancer.

The term “taxane-based chemotherapy” includes, without being limited to, docetaxel, paclitaxel, and cabazitaxel.

The term “AR-targeted therapy” means a hormone therapy, which goal is to reduce levels of male hormones, called androgens, in the body, or to stop them from fueling prostate cancer growth. The AR-targeted therapy may be i) surgical castration (orchiectomy), or ii) medical castration by administration of, without being limited to, luteinizing hormone-releasing hormone (LHRH) agonists (also called LHRH analogs or GnRH agonists) such as leuprorelin or leuprolide, goserelin, triptorelin, histrelin, nafarelin, gonadorelin, buserelin, and the like; LHRH antagonists such as degarelix, relugolix, and the like; abiraterone acetate (Zytiga®); ketoconazole; anti-androgens such as flutamide, nilutamide, bicalutamide, enzalutamide, apalutamide, darolutamide, and the like; and other androgen-suppressing drugs such as estrogens, diethylstilbestrol, and the like.

The term “line 2+” means patients with mCRPC having received already at least the two different therapies to treat the prostate cancer: taxane-based chemotherapy, and androgen receptor (AR)-targeted therapy; prior to the administration of niraparib. This term also includes patients with mCRPC having received a second, third, a fourth, or a fifth line of therapy, prior to the administration of niraparib.

The term “objective response rate” or “ORR” means the proportion of subjects or patients (both terms “subject” and “patient” used interchangeably) with BRCA DNA-repair anomalies and measurable disease whose best response is either complete response or partial response as defined by RECIST 1.1 (Eisenhauer et al 2009) and who have no evidence of bone progression according to the PCWG3 criteria (Scher et al 2016).

The term “Objective response rate in Non-BRCA Analysis Set” means the Objective response rate of soft tissue (visceral or nodal disease) as defined by RECIST 1.1 with no evidence of bone progression according to the PCWG3 criteria in subjects with measurable mCRPC and DNA-repair anomalies in ATM FANCA, PALB2, CHEK2, BRIP1, or HDAC2.

The term “CTC Response Rate” means the proportion of subjects with baseline CTC>0 whose CTC=0 per 7.5 mL blood at 8 weeks post-baseline.

The term “overall survival” or “OS” refers to the time from enrollment to the date of death from any cause. Subjects alive at time of analysis are censored on the last date the subject was known to be alive.

The term “radiographic progression” is determined by first occurrence of progression, as assessed by the investigator. Radiographic progression and bone progression were evaluated according to RECIST 1.1 for soft tissue disease and PCWG3 for bone disease as follows:
Progression of soft tissue lesions measured by CT or MRI as defined in RECIST 1.1.
Progression by bone lesions observed by bone scan and based on PCWG3. Bone progression was defined as one of the following, depending on what was observed at the Week 8 scan and what was observed on the confirmatory scan (which was performed ≥6 weeks later):
   1) Subject whose Week 8 scan was observed to have ≥2 new bone lesions compared with the baseline scan fell into one of the two categories below:
      a) Subject whose confirmatory scan showed ≥2 new lesions compared with the Week 8 scan (i.e., a total of ≥4 new lesions compared with the baseline scan) was considered to have bone scan progression at Week 8.
      b) Subject whose confirmatory scan did not show ≥2 new lesions compared with the Week 8 scan was not considered to have bone scan progression. The Week 8 scan was considered as the bone scan to which subsequent scans were compared. The first scan timepoint that showed ≥2 new lesions compared with the Week 8 scan was considered as the bone scan progression timepoint if these new lesions were confirmed by a subsequent scan 6 weeks later.
   2) For a subject whose Week 8 scan did not have ≥2 new bone lesions compared to baseline scan, the first scan timepoint that showed ≥2 new lesions compared with the Week 8 scan was considered as the bone scan progression timepoint if these new lesions were confirmed by a subsequent scan ≥6 weeks later.

Subjects without radiographic progression or death are censored at the last disease assessment date if they never started subsequent anti-cancer therapy. Subjects who began a subsequent anti-cancer therapy are censored at the date of the last assessment prior to starting a new anti-cancer therapy.

The term “progression-free survival” or “PFS” means the time from treatment enrollment to investigator-assessed disease progression (PSA, radiographic (rPFS), symptomatic, or any combination) during anti-cancer therapy or death (any cause) prior to the start of the subsequent anti-cancer therapy, whichever occurs first.

The term “time to radiographic progression” means time from enrollment to radiographic progression (as determined by the investigator) due to disease progression.

The term “time to PSA progression” means time from enrollment to the first date of documented PSA progression based on PCWG3 criteria. Subjects with no PSA progression at the time of analysis are censored on the last known date with no progression. Subjects without a baseline PSA or without any post baseline values are censored on enrollment date.

The term “time to symptomatic skeletal event” or “time to SSE” means the time from enrollment to first occurrence of one of the following symptomatic skeletal events: i) tumor-related spinal cord compression; ii) radiation to bone to relieve skeletal symptoms; iii) surgery to bone or need for tumor-related orthopedic surgical intervention; iv) symptomatic fracture or pathologic. Subjects with no symptomatic skeletal event at the time of analysis are censored on the last treatment date+30 days. Death is not considered to be an event for SSE.

The term “duration of objective response” means time from complete response or partial response to radiographic progression of disease, unequivocal clinical progression or death, whichever occurred first.

The term “circulating tumor cells (CTC) response rate” means the proportion of subjects with baseline CTC>0 whose CTC=0 per 7.5 mL blood at 8 weeks post-baseline.

### Castration Resistant Prostate Cancer

Agents that block the action (antiandrogens) of endogenous hormones (e.g., testosterone) are highly effective and routinely used for the treatment of prostate cancer (androgen ablation therapy). While initially effective at suppressing tumor growth, these androgen ablation therapies eventually fail in almost all cases, leading to CRPC. Most, but not all, prostate cancer cells initially respond to androgen withdrawal therapy like bicalutamide; this response is much less for patients treated with novel hormonal agents. However, with time, surviving populations of prostate cancer cells emerge because they have responded to the selective pressure created by androgen ablation therapy and are now refractory to it. Not only is the primary cancer refractory to available therapies, but cancer cells may also break away from the primary tumor and travel in the bloodstream, spreading the disease to distant sites (especially bone). This is known as metastatic castration resistant prostate cancer (“mCRPC”). Among other effects, this causes significant pain and further bone fragility in the subject.

### DNA Repair Genes

The subjects or patients who benefit from the improved treatment of line 2+ mCRPC are male humans over 18 years that carry biallelic anomalies in their DNA repair genes, also termed as “biallelic DNA-repair anomalies”. These anomalies may be somatic or germline. These DNA repair genes include BRCA1 (Breast Cancer gene 1), BRCA2 (Breast Cancer gene 2), ATM (ataxia-telangiectasia mutated), FANCA (Fanconi Anemia Complementation Group A gene), PALB2 (Partner and Localizer of BRCA2 gene), CHEK2 (Checkpoint Kinase 2 gene), BRIP1 (BRCA1 Interacting Protein C-terminal Helicase 1 gene), and HDAC2 (Histone deacetylase 2).

### Niraparib

Niraparib, or 2-[4-[(3S)-piperidin-3-yl]phenyl]-2H-indazole-7-carboxamide, is an orally available highly selective poly(adenosine diphosphate [ADP]-ribose) polymerase (PARP) inhibitor, with activity against PARP-1 and PARP-2 deoxyribonucleic acid (DNA)-repair polymerases. The preparation of niraparib is described in U.S. Pat. Nos. 8,071,623 and 8,436,185, both of which are incorporated herein by reference.

As used herein, the term “niraparib” means any of the free base compound (2-[4-[(3S)-piperidin-3-yl]phenyl]-2H-indazole-7-carboxamide), a salt form, including pharmaceutically acceptable salts, of 2-[4-[(3S)-piperidin-3-yl]phenyl]-2H-indazole-7-carboxamide (e.g., 4-methylbenzenesulfonic acid: 2-[4-[(3S)-piperidin-3-yl]phenyl]-2H-indazole-7-carboxamide), and/or a solvated form, including a hydrated form, thereof (e.g., 2-[4-[(3S)-piperidin-3-yl]phenyl]-2H-indazole-7-carboxamide tosylate monohydrate). Such forms may be individually referred to as “niraparib free base”, “niraparib tosylate” and “niraparib tosylate monohydrate”, respectively. Unless otherwise specified, the term “niraparib” includes all crystals, polymorphs, pseudopolymorphs, hydrates, monohydrates, anhydrous forms, solvates, salt forms, and combinations thereof, if applicable, of the compound 2-[4-[(3S)-piperidin-3-yl]phenyl]-2H-indazole-7-carboxamide. Examples of salts include, without being limited to, tosylate or 4-methylbenzenesulfonate, sulfate, benzenesulfate, fumarate, succinate, camphorate, mandelate, camsylate, and lauryl sulfate. In a particular aspect, the term “niraparib” refers to niraparib tosylate monohydrate.

The term “niraparib” also encompasses the amorphous and the crystal polymorphs of this compound, and the hydrates, ansolvates, and solvates thereof. Examples of polymorphs are described in WO 2018/183354 A1, which is incorporated herein by reference. Crystal Form I of 2-[4-[(3S)-piperidin-3-yl]phenyl]-2H-indazole-7-carboxamide tosylate monohydrate is characterized by at least one X-ray diffraction pattern reflection selected from a 2θ value of 9.5±0.2, 12.4±0.2, 13.2±0.2, 17.4±0.2, 18.4±0.2, 21.0±0.2, 24.9±0.2, 25.6±0.2, 26.0±0.2, and 26.9±0.2. Crystal Form II of 2-[4-[(3S)-piperidin-3-yl]phenyl]-2H-indazole-7-carboxamide tosylate non-stoichiometric hydrate is characterized by at least one X-ray diffraction pattern reflection selected from a 2θ value of 9.7±0.3, 12.8±0.3, 17.9±0.3, 19.7±0.3, and 21.8±0.3, Crystal Form III of 2-[4-[(3S)-piperidin-3-yl]phenyl]-2H-indazole-7-carboxamide tosylate anhydrous form is characterized by at least one X-ray diffraction pattern reflection selected from a 2θ value of 17.8±0.2, 19.0±0.2, or 22.8±0.2. Crystal Form I is preferred. More examples of polymorphs are described in WO 2020/072797 A1, which is incorporated herein by reference.

The term “niraparib eq.” or “niraparib equivalent” refers to the free base dose amount of niraparib.

The invention also provides pharmaceutical compositions comprising niraparib and a pharmaceutically acceptable carrier. The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium croscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water-soluble taste masking material such as hydroxypropyl-methylcellulose or hydroxypropylcellulose, or a time delay material such as ethyl cellulose, cellulose acetate butyrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water-soluble carrier such as polyethylene glycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally occurring phosphatides, for example soybean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant. The pharmaceutical compositions may be in the form of a sterile injectable aqueous solution. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulsion.

The injectable solutions or microemulsions may be introduced into a patient's blood stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleaginous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butanediol. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

Niraparib may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the instant compounds are employed. (For purposes of this application, topical application shall include mouth washes and gargles.)

Niraparib can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Niraparib may also be delivered as a suppository employing bases such as cocoa butter, glycerinated gelatin, hydrogenated vegetable oils, mixtures of polyethylene glycols of various molecular weights and fatty acid esters of polyethylene glycol.

### EXAMPLES

These examples are provided for illustrative purposes only and not to limit the scope of the claims provided herein.

### Example 1: A Phase 2 Efficacy Ad Safely Study of Niraparib in Men with Metastatic Castration-Resistant Prostate Cancer and DNA-Repair Anomalies—The Galahad Study

Study Center(s): Subjects were enrolled across 15 countries: Australia (8 sites), Belgium (9 sites), Brazil (sites), Canada (5 sites), Denmark (1 site), France (9 sites), Israel (4 sites), Netherlands (4 sites). Korea (3 sites), Russia (3 sites). Spain (10 sites) Sweden (4 sites), Taiwan (4 sites), United Kingdom (7 sites), United States (14 sites).

### Objectives:

| | |
|---|---|
| Objectives | Endpoints/Assessments |

Primary

| | |
|---|---|
| To assess the efficacy of niraparib in | ORR of soft tissue (visceral or nodal disease) as |
| subjects with measurable mCRPC and | defined by RECIST 1.1 with no evidence of bone |
| BRCA mutation | progression according to the PCWG3 criteria |

Secondary

| | |
|---|---|
| To assess the efficacy of niraparib in | ORR of soft tissue (visceral or nodal disease) as |
| subjects with measurable mCRPC and | defined by RECIST 1.1 with no evidence of bone |
| non-BRCA mutation | progression according to the PCWG3 criteria |
| To assess the efficacy of niraparib in | CTC response defined as CTC = 0 per 7.5 mL blood |
| subjects with mCRPC and DNA- | at 8 weeks post-baseline in subjects with baseline |
| repair anomalies | CTC >0 |
| To evaluate response outcomes of | OS: time from enrollment to death from any cause |
| niraparib in subjects with mCRPC and | As defined by PCWG3 |
| DNA-repair anomalies (BRCA, non- | rPFS: time from enrollment to radiographic |
| BRCA) | progression or death from any cause, |
| | whichever occurred first |
| | Time to radiographic progression |
| | Time to PSA progression |
| | Time to SSE |
| To evaluate the safety and tolerability | Incidence of AEs |
| of niraparib | Clinical laboratory test results |
| To evaluate duration of tumor | Duration of objective response: time from CR or PR |
| response (BRCA, non-BRCA) | to radiographic progression of disease, unequivocal |
| | clinical progression, or death, whichever occurred |
| | first |

Methodology: This was a Phase 2, multicenter, open-label study to assess the efficacy and safety of once daily dosing of 300 mg niraparib in male subjects over the age of 18 years with mCRPC and DNA repair anomalies who had received prior taxane-based chemotherapy and AR-targeted therapy. The planned sample size was approximately 120 subjects with measurable disease who were biomarker-positive (approximately 75 subjects with DNA repair anomalies in BRCA [BRCA1 or BRCA2] and approximately 45 subjects with biallelic DNA-repair anomalies in non-BRCA [ATM, FANCA, PALB2, CHEK2, BRIP1, or HDAC2]).

In addition, at least 90 subjects with non-measurable disease (i.e., bone disease only) regardless of their DNA anomaly (i.e., BRCA or non-BRCA) were included to evaluate the activity of niraparib in this population. Efficacy objectives were evaluated for subjects meeting the biomarker selection criteria described in the protocol. All subjects were monitored for safety during the study period, and up to 30 days after the last dose of study drug. Treatment continued until disease progression, unacceptable toxicity, death, or termination of the study by the sponsor.

### Study Population

Number of Subjects (planned and analyzed): Enrollment of 120 biomarker-positive subjects with measurable disease was planned for this study. In addition, at least 90 subjects with non-measurable disease (i.e., bone disease only) were also to be included.

In this analysis, a total of 289 subjects who enrolled in the study and received at least 1 dose of niraparib were included in the safety population. The biallelic/germline population used for the efficacy analyses consists of 223 subjects with biallelic DRD loss. Of the 223 subjects, 142 have BRCA DRD and 81 have non-BRCA DRD.

Diagnosis and Main Criteria for Inclusion: The target population consisted of male subjects over the age of 18 years with mCRPC and DNA-repair anomalies (biallelic mutations and germline pathogenic mutations only for BRCA1 or BRCA2 mutations) who had received at least 1 prior taxane-based chemotherapy and at least 1 prior AR targeted therapy (second-generation or later). Subjects were asked to provide a tumor tissue sample (archival or recently collected, if available), and a blood sample for analysis of DNA-repair anomalies using a sponsor-validated assay.

Subjects must had demonstrated evidence of disease progression on or after treatment with taxane-based chemotherapy and a second-generation AR-targeted therapy for metastatic prostate cancer; or had discontinued taxane-based chemotherapy due to an AE.

In the setting of castrate levels of testosterone in subjects on a GnRHa or due to bilateral orchiectomy, progression of metastatic prostate cancer had to be demonstrated by PSA progression, or radiographic progression of soft tissue by RECIST 1.1 or bone disease by PCWG3 criteria.

Subjects were required to continue gonadotropin releasing hormone analogs during the course of the study if not surgically castrated.

### Prescreening Eligibility Criteria

Signed prescreening ICF.
Willing to provide a tumor tissue sample (archival or recently collected), and a blood sample for analysis of DNA-repair anomalies using a sponsor-validated assay.
Must have received or been on at least 1 taxane-based chemotherapy for metastatic prostate cancer and at least 1 AR-targeted (second-generation or later) therapy.

### Inclusion Criteria

Subjects enrolled in this study were required to meet the following key acceptance criteria:
Histologically confirmed prostate cancer (mixed histology was acceptable, with the exception of the small cell pure phenotype, which was excluded).
Have received a taxane-based chemotherapy for the treatment of metastatic prostate cancer with evidence of disease progression on or after treatment, or discontinued from a taxane-based chemotherapy due to an adverse event.
Have received a second-generation or later AR-targeted therapy (for example, abiraterone acetate plus prednisone, enzalutamide, apalutamide) for the treatment of metastatic prostate cancer with evidence of disease progression, or non-mCRPC with evidence of subsequent metastasis.
Biomarker-positive by at least one of the following criteria.
   Biallelic DNA-repair anomaly based on a sponsor validated blood or tissue assay.
   Germline pathogenic BRCA1 or BRCA2 by any test (somatic local results must have been confirmed as positive by the sponsor-validated assay before dosing).
Progression of metastatic prostate cancer in the setting of castrate levels of testosterone ≤50 ng/dL on a GnRHa, or history of bilateral orchiectomy at study entry, as defined in the protocol.
Able to continue GnRHa during the course of the study if not surgically castrated.

### Exclusion Criteria

Subjects were not to be enrolled into the study if it were determined upon pre-study examination that they met the following key criteria:
Prior treatment with a PARP inhibitor.
Prior platinum-based chemotherapy for the treatment of prostate cancer.
Known history or current diagnosis of MDS/AML.
Symptomatic or impending cord compression, except if subject had received definitive treatment for this and demonstrated evidence of clinically stable disease.
Symptomatic brain metastases.
Known allergies, hypersensitivity, or intolerance to niraparib or its excipients.

TABLE 1
Summary of Prostate Cancer Baseline Clinical
Disease Characteristics; ITT Analysis Set

| | Niraparib | |
|---|---|---|
| Analysis set: ITT | 223 | |
| Extent of disease | | |
| N | 223 | |
| Bone | 206 | (92.4%) |
| Visceral | 53 | (23.8%) |
| Liver | 37 | (16.6%) |
| Lung | 25 | (11.2%) |
| Lymph Node | 118 | (52.9%) |
| Soft Tissue | 38 | (17.0%) |
| Evidence of disease progression | | |
| N | 222 | |
| PSA and Radiographic | 164 | (73.9%) |
| PSA only | 44 | (19.8%) |
| Radiographic only | 14 | (6.3%) |
| ECOG Performance status score | | |
| N | 223 | |
| 0 | 66 | (29.6%) |
| 1 | 125 | (56.1%) |
| 2 | 32 | (14.3%) |
| Gleason Score at initial diagnosis | | |
| N | 212 | |
| <7 | 13 | (6.1%) |
| 7 | 52 | (24.5%) |
| 3 + 4 | 23 | (10.8%) |
| 4 + 3 | 26 | (12.3%) |
| U + U | 3 | (1.4%) |
| >=8 | 147 | (69.3%) |
| Number of prior therapies for prostate cancer | | |
| Number of AR-targeted, taxane, cytotoxic chemotherapy, | | |
| and other therapy | | |
| N | 223 | |
| 2 | 81 | (36.3%) |
| 3 | 85 | (38.1%) |
| 4 | 40 | (17.9%) |
| 5 | 16 | (7.2%) |
| 6 | 1 | (0.4%) |
| Number of taxane-based chemotherapy | | |
| N | 223 | |
| 1 | 141 | (63.2%) |
| 2 | 82 | (36.8%) |
| Number of AR-targeted therapy | | |
| N | 223 | |
| 1 | 142 | (63.7%) |
| Number of taxane-based chemotherapy | | |
| 1 | 95 | (42.6%) |
| 2 | 47 | (21.1%) |
| 2 | 75 | (33.6%) |
| Number of taxane-based chemotherapy | | |
| 1 | 42 | (18.8%) |
| 2 | 33 | (14.8%) |
| 3 | 6 | (2.7%) |
| Number of taxane-based chemotherapy | | |
| 1 | 4 | (1.8%) |
| 2 | 2 | (0.9%) |

Key: BPI-SF = Brief Pain Inventory - Short Form, ECOG = Eastern Cooperative Oncology Group, PSA = prostate-specific antigen, WHO = World Health Organization
ITT: BRCA or non BRCA subjects; BRCA: biallelic BRCA1 or BRCA2 or germline BRCA; non-BRCA; biallelic in ATM, FANCA, PALB2, CHEK2, BRIP1 or HDAC2

TABLE 2
Summary of Baseline Laboratory Parameters; ITT Analysis Set

| | Niraparib | |
|---|---|---|
| Analysis set: ITT | 223 | |
| PSA (ng/mL) | | |
| N | 223 | |
| Mean (SD) | 579.5 | (1250.32) |
| Median | 147.5 | |
| Range | (0; 11370) | |
| Hemoglobin (g/L) | | |
| N | 223 | |
| Mean (SD) | 115.0 | (13.82) |
| Median | 116.0 | |
| Range | (83; 148) | |
| CTC (/7.5mL) | | |
| N | 212 | |
| Mean (SD) | 109.6 | (282.31) |
| Median | 32.0 | |
| Range | (0; 3137) | |
| Missing | 11 | (4.9%) |
| =0 | 10 | (4.5%) |
| >0 to <5 | 25 | (11.2%) |
| >=5 | 177 | (79.4%) |
| Platelet (x10E9/L) | | |
| N | 223 | |
| Mean (SD) | 273.4 | (99.86) |
| Median | 257.0 | |
| Range | (53; 629) | |
| Neutrophils, Segmented (x10E9/L)^{a} | | |
| N | 223 | |
| Mean (SD) | 4.8 | (2.00) |
| Median | 4.5 | |
| Range | (1; 13) | |
| Testosterone (nmol/L) | | |
| N | 221 | |
| Mean (SD) | 1.2 | (0.33) |

| Median | 12 |
|---|---|
| Range | (0; 6) |

Note:
All subjects met the study inclusion and exclusion criteria for baseline laboratory parameters at the Screening visit. Values presented in this table are the values closest to Cycle 1, Visit 1 and may be different to Screening values.
^{a}Includes Neutrophils (x10E9/L) if the value of Neutrophils, Segmented (x10E9/L) is missing.
ITT: BRCA or non BRCA subjects; BRCA: biallelic BRCA1 or BRCA2 or germline BRCA; non-BRCA; biallelic in ATM, FANCA, PALB2, CHEK2, BRIP1 or HDAC2

### Dosage and Administration

Test Product, Dose and Mode of Administration: Subjects received 300 mg niraparib as 3×100 mg capsules for once daily oral administration.

Duration of Treatment: Treatment began at Cycle 1 Day 1 in the Treatment Phase and continued in 28-day cycles until the study drug was discontinued.

TABLE 3
Summary of Exposure to Study Agent; ITT Analysis Set

| | Niraparib |
|---|---|
| Analysis set: ITT | 223 |
| Total number of cycles started | |

| | | | |
|---|---|---|---|
| >=1 | cycle | 223 | (100.0%) |
| >=2 | cycles | 205 | (91.9%) |
| >=3 | cycles | 177 | (79.4%) |
| >=4 | cycles | 151 | (67.7%) |
| >=5 | cycles | 131 | (58.7%) |
| >=6 | cycles | 117 | (52.5%) |
| >=7 | cycles | 97 | (43.5%) |
| >=8 | cycles | 84 | (37.7%) |
| >=9 | cycles | 70 | (31.4%) |
| >=10 | cycles | 50 | (22.4%) |
| >=11 | cycles | 41 | (18.4%) |
| >=12 | cycles | 32 | (14.3%) |
| 13-18 | cycles | 15 | (6.7%) |
| 19-24 | cycles | 3 | (1.3%) |

| 25 or more cycles | 2 | (0.9%) |
|---|---|---|
| Total number of cycles started | | |
| N | 223 | |
| Mean (SD) | 6.62 | (4.754) |
| Median | 6.00 | |
| Range | (1.0; 30.0) | |
| Total duration of exposure (months)^{a} | | |

| | | | |
|---|---|---|---|
| >=1 | month | 207 | (92.8%) |
| >=2 | months | 172 | (77.1%) |
| >=3 | months | 152 | (68.2%) |
| >=4 | months | 128 | (57.4%) |
| >=5 | months | 116 | (52.0%) |
| >=6 | months | 98 | (43.9%) |
| >=7 | months | 79 | (35.4%) |
| >=8 | months | 62 | (27.8%) |
| >=9 | months | 46 | (20.6%) |
| >=10 | months | 38 | (17.0%) |
| >=11 | months | 29 | (13.0%) |
| >=12 | months | 14 | (6.3%) |
| 13-18 | months | 8 | (3.6%) |
| 19-24 | months | 2 | (0.9%) |

| 25 or more months | 1 | (0.4%) |
|---|---|---|
| Total duration of exposure (months)^{a} | | |
| N | 223 | |
| Mean (SD) | 5.88 | (4.409) |

| Median | 5.49 |
|---|---|
| Range | (0.2; 27.0) |

^{a}Total duration of exposure is defined as (date of last dose of study agent − date of first dose of study agent) + 1 divided by 30.4375.
ITT: BRCA or non BRCA subjects; BRCA: biallelic BRCA1 or BRCA2 or germline BRCA; non-BRCA; biallelic in ATM, FANCA, PALB2, CHEK2, BRIP1 or HDAC2

### Study Evaluations

Criteria for Evaluation: Efficacy assessments included chest, abdomen, and pelvis CT or MRI scans and whole-body bone scans (^{99m}Tc) to evaluate objective response and disease progression.

Serum PSA, CTCs, survival status, SSEs were also collected. PROs included the BPI-SF, FACT-P, and EQ-5D-5L questionnaires. Safety assessments included recording of AEs, physical examinations, vital signs, ECGs, and ECOG performance status. Clinical laboratory testing included hematology, blood chemistry, and liver function parameters. Blood samples were also taken for pharmacokinetic assessments and biomarker analysis. Archival or recently collected tumor tissue samples were obtained from consenting subjects for biomarker identification.

Statistical Methods: Efficacy analyses were performed on the ITT population, which included subjects who had received at least 1 dose of study drug and had BRCA (biallelic or germline DNA-repair anomalies) or non-BRCA (biallelic DNA-repair anomaly). The primary endpoint was ORR and summarized for the BRCA analysis set along with the 95% 2-sided exact CI. In addition, the counts and percentage of subjects in each response category (CR. PR, etc.) were tabulated. ORR in non-BRCA subjects was analyzed in the same way.

Descriptive summaries of CTC response were generated for BRCA and non-BRCA analysis sets, measurable and non-measurable subgroups and tabulated with its 2-sided 95% exact CI. Waterfall plots of percent change in CTC were also presented, showing the percentage change in CTC counts from baseline to 8 weeks, as well as maximal CTC count declines. All time to event secondary endpoints were evaluated using Kaplan-Meier method for BRCA and non-BRCA analysis sets. Median time to event and the corresponding 95% CI were provided. Descriptive summaries were provided for BRCA and non-BRCA analysis sets. In addition, waterfall plots for PSA were presented to demonstrate the percentage of change in PSA from baseline to 12 weeks (or earlier for those who discontinued therapy), as well as the maximal decline in PSA.

Descriptive statistics (N, Mean, Standard Deviation, Median, Min. Max for observed and changes from baseline) were provided for each component of the BPI-SF, FACT-P and EQ-5D-5L. Time to degradation was determined using the following meaningful change threshold values: FACT-P Total (10-point reduction from baseline), EQ-5D-5L Index (0.09-point reduction from baseline). EQ-5D-5L VAS (10-point reduction from baseline), and BPI SF worst pain intensity item (30% reduction from baseline).

All AEs reported on or after the date of first dose until 30 days (inclusive) after the last dose of study drug were considered treatment-emergent and were summarized. AE incidence rates were summarized with frequency and percentage by SOC and Preferred Term, with all subjects treated as the denominator, unless otherwise specified. In addition. AE incidence rates were also summarized by severity and relationship to study drug. Treatment-related AEs were those judged by the investigator to beat least possibly related to the study drug. Subjects with multiple occurrences of events were only counted once at the maximum severity to study drug for each preferred team, SOC, and overall. Deaths that occurred within 30 days after the last dose of study drug were defined as on-study deaths. No inferential statistical analyses were performed in analyzing the safety data.

### Primary Endpoint

The primary endpoint was ORR, defined as the proportion of subjects with BRCA DNA-repair anomalies and measurable disease whose best response was either CR or PR as defined by RECIST 1.1 (Eisenhauer et al 2009) and who had no evidence of bone progression according to the PCWG3 criteria (Scher et al 2016).

The primary estimand, the main clinical quantity of interest to be estimated in this study, was defined by the following 4 components:
Population: all subjects with measurable disease in the BRCA analysis set
Variable: ORR based on investigator assessment
Intercurrent events and strategies:

| | |
|---|---|
| Missing baseline assessment | Assume non-response |
| No post baseline response assessment | Assume non-response |

Population-level summary: ORR was summarized for the BRCA analysis set along with 95% 2-sided exact CI.

ORR final analysis was performed approximately 6 months from Cycle 1 Day 1 of the last subject with a BRCA DNA-repair anomaly and measurable disease. Objective response as determined by investigator assessment was considered as the primary analysis.

Additionally, all enrolled subjects with a non-BRCA DNA-repair anomaly and measurable disease as defined by RECIST 1.1 (Eisenhauer et al 2009) were analyzed for objective response.

Subjects who discontinued the study without a response assessment were considered as non-responders in the analysis. The objective response rate was calculated and its 2-sided 95% exact CI presented. In addition, the counts and percentage of subjects in each response category (CR, PR, etc.) were tabulated.

### Secondary Endpoints

Secondary endpoints included ORR in non-BRCA analysis set, CTC response rate, OS, rPFS, time to radiographic progression, time to PSA progression, time to SSE, and duration of objective response.

### Secondary Endpoint Analysis Methods:

ORR in BRCA and non-BRCA subjects were analyzed in the same way.

The following analyses of CTC response were performed for BRCA and non-BRCA analysis sets, measurable and non-measurable subgroups:
Descriptive summaries
CTC response rate was tabulated with its 2-sided 95% exact CI

Waterfall plots of percent change in CTC were also presented, showing the percentage change in CTC counts from baseline to 8 weeks, as well as maximal CTC count declines.

All time to event secondary endpoints were evaluated using Kaplan-Meier method for BRCA and non-BRCA analysis sets. Median time to event and the corresponding 95% CI were provided. Descriptive summaries were provided for BRCA and non-BRCA analysis sets. In addition, waterfall plots for PSA were presented to demonstrate the percentage of change in PSA from baseline to 12 weeks (or earlier for those who discontinued therapy), as well as the maximal decline in PSA.

### Results

### Study Population

All of the 289 subjects enrolled into the study (100%) were treated with 300 mg niraparib and included in the safety population. The median duration of follow-up was 10 months (range 0.3 to 47 months) in the enrolled analysis population. At the time of clinical data cutoff (26 Jan. 2021), 94% of subjects had prematurely discontinued niraparib treatment. The most common reason for treatment discontinuation was progressive disease (71%), followed by adverse events (14%). After treatment discontinuation, subjects continued to be followed on study for survival, disease evaluations and SSEs. At the time of analysis, 6% of subjects were still on study (including subjects who had entered the long-term extension) and 94% subjects had prematurely terminated study participation. Death was the main reason for premature study discontinuation (72% of subjects).

The majority of subjects were white (70%), with a median age of 69 years (range 46 to 88 years). Subjects in the primary efficacy analysis population (measurable BRCA ITT) were slightly younger, with a median age of 66 years (range 47 to 86 years). Demographics in the enrolled, BRCA ITT, and non-BRCA ITT populations were comparable to the ITT population.

All subjects had evidence of disease progression at study entry; 74% of subjects had both PSA and radiographic progression at entry. Most subjects had an ECOG performance status score of either 0 (30%) or 1 (56%) at baseline. The majority of subjects (69%) had a Gleason score of 8 or greater. Almost all subjects (92%) had bone metastases and 24% of subjects had visceral disease, primarily driven by liver metastases. For subjects with measurable disease and BRCA DRD, the incidence of visceral disease was even higher (30 of 76 subjects [40%]). All other baseline disease characteristics were comparable across the analysis populations.

### Results

Treatment with 300 mg niraparib resulted in an ORR of 34.2% (95% CI: 23.7 to 46.0) in BRCA subjects with measurable disease.
Complete response was observed in 2 BRCA subjects with measurable disease; in one subject the response was maintained for 9.7 months, in the other subject the response lasted a total of 9.5 months.
CTC response rate was 24% in BRCA subjects compared with a rate of 9% in non-BRCA subjects, and 18% in the combined groups.
Median OS was 12 months in the overall ITT population, with 153 events occurring (69% of ITT subjects). A more favorable survival rate was observed in BRCA subjects (13 months) compared with non-BRCA subjects (10 months).
Median rPFS was 5.6 months in the overall ITT population, with 144 events occurring in 65% of subjects. In the 142 BRCA subjects, the median rPFS was longer at 8.1 months, with 87 events occurring in 61% of BRCA subjects.
Time to radiographic progression was 5.8 months in the ITT population, with 134 events occurring in 60% of subjects. Time to radiographic progression was slightly longer in BRCA subjects, with 81 events in 57% of BRCA subjects.
Median time to PSA progression was reached at 4.6 months in the ITT population (124 events, 56% of subjects) and was similar in BRCA subjects (85 events, 60% of subjects).
Sixty-five events in 29% of subjects were recorded in the ITT population. The incidence of symptomatic skeletal events was comparable in BRCA subjects, with 46 events in 32% of subjects. Median time to symptomatic skeletal event was approximately 13 months in the overall ITT and BRCA ITT populations.
In the 31 subjects with measurable disease who responded to niraparib treatment, median duration of objective response was 5.55 months (range: 3.91 to 7.20). Response durations were similar regardless of genetic mutation.
Compliance with PRO assessments was high. FACT-P, and EQ-5D-5L VAS and Index scores appeared stable over time and similar between groups.

TABLE 4
Summary of Objective Response per INV Based on RECIST 1.1
Criteria and Bone Progression Criteria in Subjects with Measurable
Disease at Baseline; Measurable ITT Analysis Set

| | Niraparib | | |
|---|---|---|---|
| | BRCA | NON-BRCA | ALL |
| Analysis set: Measurable ITT | 76 | 47 | 123 |
| Objective response confirmed | | | |
| Complete Response (CR) | 2 (2.6%) | 0 | 2 (1.6%) |
| Partial Response (PR) | 24 (31.6%) | 5 (10.6%) | 29 (23.6%) |
| ORR (95% CI) | 26 (34.2%) | 5 (10.6%) | 31 (25.2%) |
| | (23.7%, 46.0%) | (3.5%, 23.1%) | (17.8%, 33.8%) |

ITT: BRCA or non BRCA subjects; BRCA: biallelic BRCA1 or BRCA2 or germline BRCA; non-BRCA; biallelic in ATM, FANCA, PALB2, CHEK2, BRIP1 or HDAC2

TABLE 5
Summary of Best Overall Response per INV Based on RECIST 1.1
Criteria and Bone Progression Criteria in Subjects with Measurable
Disease at Baseline; Measurable ITT Analysis Set

| | Niraparib | | | |
|---|---|---|---|---|
| | BRCA | NON-BRCA | | ALL |
| Analysis set: Measurable ITT | 76 | 47 | | 123 |
| Best Overall Response | | | | |
| Complete Response (CR) | 2 (2.6%) | 0 | | 2 (1.6%) |
| Partial Response (PR) | 24 (31.6%) | 5 | (10.6%) | 29 (23.6%) |
| Stable Disease (SD) | 27 (35.5%) | 19 | (40.4%) | 46 (37.4%) |
| Progressive Disease (PD) | 16 (21.1%) | 15 | (31.9%) | 31 (25.2%) |
| Not Available (NA) | 7 (9.2%) | 8 | (17.0%) | 15 (12.2%) |

ITT: BRCA or non BRCA subjects; BRCA: biallelic BRCA1 or BRCA2 or germline BRCA; non-BRCA; biallelic in ATM, FANCA, PALB2, CHEK2, BRIP1 or HDAC2

TABLE 6
CTC Response; ITT Analysis Set

| | Niraparib | | | | |
|---|---|---|---|---|---|
| | BRCA | | NON-BRCA | ALL | |
| Analysis set: ITT | 142 | | 81 | 223 | |
| CTC (/7.5 mL) | | | | | |
| Baseline >0 | 131 | (92.3%) | 71 (87.7%) | 202 | (90.6%) |
| CTC Response (CTC = | 31 | (23.7%) | 6 (8.5%) | 37 | (18.3%) |
| 0 at week 8) | | | | | |

ITT: BRCA or non BRCA subjects; BRCA: biallelic BRCA1 or BRCA2 or germline BRCA; non-BRCA; biallelic in ATM, FANCA, PALB2, CHEK2, BRIP1 or HDAC2

TABLE 7
Summary of Overall Survival; ITT Analysis Set

| | Niraparib | | | | | |
|---|---|---|---|---|---|---|
| | BRCA | | NON-BRCA | | ALL | |
| Analysis set: ITT | 142 | | 81 | | 223 | |
| Event | 88 | (62.0%) | 65 | (80.2%) | 153 | (68.6%) |
| Censored | 54 | (38.0%) | 16 | (19.8%) | 70 | (31.4%) |
| Time to event (months) | | | | | | |
| 25th percentile (95% CI) | 8.31 | (6.51, 9.49) | 4.86 | (3.71, 7.13) | 7.16 | (5.45, 8.25) |
| Median (95% CI) | 13.01 | (11.04, 14.29) | 9.63 | (8.05, 13.44) | 12.29 | (10.09, 13.31) |
| 75th percentile (95% CI) | 19.52 | (17.61, 24.41) | 16.53 | (13.96, 20.07) | 19.42 | (16.39, 20.76) |
| Range | (0.3, 47.4*) | | (0.4*, 35.3) | | (0.3, 47.4*) | |
| 6-month event-free rate (95% CI) | 0.841 | (0.769, 0.892) | 0.709 | (0.596, 0.796) | 0.793 | (0.733, 0.841) |
| 12-month event-free rate (95% CI) | 0.564 | (0.472, 0.646) | 0.413 | (0.300, 0.522) | 0.508 | (0.437, 0.576) |
| 18-month event-free rate (95% CI) | 0.334 | (0.240, 0.431) | 0.198 | (0.109, 0.305) | 0.278 | (0.208, 0.351) |
| 24-month event-free rate (95% CI) | 0.152 | (0.077, 0.251) | 0.111 | (0.044, 0.212) | 0.136 | (0.081, 0.206) |

Note:
*= censored observation, NE = not estimable
ITT: BRCA or non BRCA subjects; BRCA: biallelic BRCA1 or BRCA2 or germline BRCA; non-BRCA; biallelic in ATM, FANCA, PALB2, CHEK2, BRIP1 or HDAC2

TABLE 8
Summary of rPFS; ITT Analysis Set

| | Niraparib | | | | | |
|---|---|---|---|---|---|---|
| | BRCA | | NON-BRCA | | ALL | |
| Analysis set: ITT | 142 | | 81 | | 223 | |
| Event | 87 | (61.3%) | 57 | (70.4%) | 144 | (64.6%) |
| Censored | 55 | (38.7%) | 24 | (29.6%) | 79 | (35.4%) |
| Time to event (months) | | | | | | |
| 25th percentile (95% CI) | 4.44 | (2.79, 5.42) | 1.87 | (1.81, 1.91) | 2.07 | (1.87, 3.68) |
| Median (95% CI) | 8.08 | (5.55, 8.38) | 3.71 | (1.97, 5.49) | 5.59 | (5.42, 7.59) |
| 75th percentile (95% CI) | 11.56 | (10.58, 13.80) | 6.47 | (5.55, 10.71) | 11.01 | (8.38, 11.56) |
| Range | (0.0*, 22.0*) | | (0.0*, 11.4*) | | (0.0*, 22.0*) | |
| 6-month event-free rate (95% CI) | 0.572 | (0.479, 0.655) | 0.289 | (0.180, 0.407) | 0.475 | (0.401, 0.545) |
| 12-month event-free rate (95% CI) | 0.215 | (0.129, 0.316) | NE | (NE, NE) | 0.155 | (0.093, 0.232) |
| 18-month event-free rate (95% CI) | 0.043 | (0.004, 0.165) | NE | (NE, NE) | 0.031 | (0.003, 0.123) |
| 24-month event-free rate (95% CI) | NE | (NE, NE) | NE | (NE, NE) | NE | (NE, NE) |

Note:
*= censored observation, NE = not estimable
ITT: BRCA or non BRCA subjects; BRCA: biallelic BRCA1 or BRCA2 or germline BRCA; non-BRCA; biallelic in ATM, FANCA, PALB2, CHEK2, BRIP1 or HDAC2

TABLE 9
Summary of Time to Radiographic Progression; ITT Analysis Set

| | Niraparib | | | | | |
|---|---|---|---|---|---|---|
| | BRCA | | NON-BRCA | | ALL | |
| Analysis set: ITT | 142 | | 81 | | 223 | |
| Event | 81 | (57.0%) | 53 | (65.4%) | 134 | (60.1%) |
| Censored | 61 | (43.0%) | 28 | (34.6%) | 89 | (39.9%) |
| Time to event (months) | | | | | | |
| 25th percentile (95% CI) | 4.76 | (3.19, 5.45) | 1.87 | (1.81, 1.91) | 2.76 | (1.91, 3.71) |
| Median (95% CI) | 8.08 | (5.75, 8.97) | 3.78 | (2.00, 5.55) | 5.78 | (5.49, 8.08) |
| 75th percentile (95% CI) | 11.63 | (11.01, 15.67) | 8.11 | (5.55, 10.71) | 11.07 | (8.97, 12.29) |
| Range | (0.0*, 22.0*) | | (0.0*, 11.4*) | | (0.0*, 22.0*) | |
| 6-month event-free rate | 0.593 | (0.499, 0.676) | 0.299 | (0,186, 0,421) | 0.494 | (0.418, 0.565) |
| (95% CI) | | | | | | |
| 12-month event-free rate | 0.234 | (0.141, 0.341) | NE | (NE, NE) | 0.170 | (0.103, 0.253) |
| (95% CI) | | | | | | |
| 18-month event-free rate | 0.046 | (0.004, 0.177) | NE | (NE, NE) | 0.034 | (0.003, 0.134) |
| (95% CI) | | | | | | |
| 24-month event-free rate | NE | (NE, NE) | NE | (NE. NE) | NE | (NE, NE) |
| (95% CI) | | | | | | |

Note:
*= censored observation, NE = not estimable
ITT: BRCA or non BRCA subjects; BRCA: biallelic BRCA1 or BRCA2 or germline BRCA; non-BRCA; biallelic in ATM, FANCA, PALB2, CHEK2, BRIP1 or HDAC2

TABLE 10
Summary of Time to PSA Progression; ITT Analysis Set

| | Niraparib | | | | | |
|---|---|---|---|---|---|---|
| | BRCA | | NON-BRCA | | ALL | |
| Analysis set: ITT | 142 | | 81 | | 223 | |
| Event | 85 | (59.9%) | 39 | (48.1%) | 124 | (55.6%) |
| Censored | 57 | (40.1%) | 42 | (51.9%) | 99 | (44.4%) |
| Time to event (months) | 3.48 | (2.79, 3.78) | 2.79 | (2.79, 2.83) | 2.83 | (2.79, 3.15) |
| 25th percentile (95% CI) | | | | | | |
| Median (95% CI) | 5.13 | (4.60, 5.59) | 3.65 | (2.83, 3.71) | 4.63 | (3.78, 5.13) |
| 75th percentile (95% CI) | 11.07 | (6.67, 11.14) | 4.63 | (3.71, 8.31) | 8.31 | (6.41, 11.07) |
| Range | (0.0*, 13.7*) | | (0.0*, 8.3) | | (0.0*, 13.7*) | |
| 6-month event-free rate | 0.393 | (0.300, 0.485) | 0.144 | (0.043, 0.303) | 0.328 | (0.252, 0.406) |
| (95% CD) | | | | | | |
| 12-month event-free rate | 0.067 | (0.015, 0.175) | 0.000 | (NE, NE) | 0.053 | (0.012, 0.141) |
| (95% CI) | | | | | | |
| 18-month event-free rate | NE | (NE, NE) | 0.000 | (NE, NE) | NE | (NE, NE) |
| (95% CI) | | | | | | |
| 24-month event-free rate | NE | (NE, NE) | 0.000 | (NE, NE) | NE | (NE, NE) |
| (95% CI) | | | | | | |

Note:
*= censored observation, NE = not estimable
ITT: BRCA or non BRCA subjects; BRCA: biallelic BRCA1 or BRCA2 or germline BRCA; non-BRCA; biallelic in ATM, FANCA, PALB2, CHEK2, BRIP1 or HDAC2

TABLE 11
Summary of Time to Symptomatic Skeletal Event; ITT Analysis Set

| | Niraparib | | | | | |
|---|---|---|---|---|---|---|
| | BRCA | | NON-BRCA | | ALL | |
| Analysis set: ITT | 142 | | 81 | | 223 | |
| Event | 46 | (32.4%) | 19 | (23.5%) | 65 | (29.1%) |
| Censored | 96 | (67.6%) | 62 | (76.5%) | 158 | (70.9%) |
| Time to event (months) | | | | | | |
| 25th percentile (95% CI) | 7.52 | (6.01, 8.90) | 6.51 | (4.83, 9.95) | 6.97 | (6.01, 8.54) |
| Median (95% CI) | 13.80 | (10.41, NE) | 10.35 | (8.18, NE) | 12.88 | (10.35, 17.54) |
| 75th percentile (95% CI) | NE | (14.52, NE) | 15.54 | (10.35, NE) | NE | (15.54, NE) |
| Range | (0.1, 28.0*) | | (0.0, 23.5*) | | (0.0, 28.0*) | |
| 6-month event-free rate (95% CI) | 0.844 | (0.765, 0.898) | 0.792 | (0.642, 0.885) | 0.830 | (0.764, 0.879) |
| 12-month event-free rate (95% CI) | 0.543 | (0,423, 0.648) | 0.379 | (0.149, 0,610) | 0.511 | (0.404, 0.608) |
| 18-month event-free rate (95% CI) | 0.323 | (0.146, 0.514) | 0.190 | (0.015, 0.516) | 0.297 | (0.148, 0.463) |
| 24-month event-free rate (95% CI) | 0.323 | (0.146, 0.514) | NE | (NE, NE) | 0.297 | (0.148, 0.463) |

Note:
*= censored observation, NE = not estimable
ITT: BRCA or non BRCA subjects; BRCA: biallelic BRCA1 or BRCA2 or germline BRCA; non-BRCA; biallelic in ATM, FANCA, PALB2, CHEK2, BRIP1 or HDAC2

TABLE 12
Summary of Duration of Objective Response per INV by
RECIST 1.1; Measurable ITT Responder Analysis Set

| | Niraparib | | | | | |
|---|---|---|---|---|---|---|
| | BRCA | | NON-BRCA | | ALL | |
| Analysis set: Measurable ITT | 26 | | 5 | | 31 | |
| Responder | | | | | | |
| Event | 20 | (76.9%) | 4 | (80.0%) | 24 | (77.4%) |
| Censored | 6 | (23.1%) | 1 | (20.0%) | 7 | (22.6%) |
| Time to event (months) | | | | | | |
| 25th percentile (95% CI) | 3.75 | (3.38, 4.63) | 2.96 | (2.14, 6.54) | 3.75 | (3.38, 4.50) |
| Median (95% CI) | 5.55 | (3.91, 7.20) | 5.16 | (2.14, NE) | 5.55 | (3.78, 6.54) |
| 75th percentile (95% CI) | 7.20 | (6.28, 9.72) | 6.95 | (2.14, NE) | 7.20 | (6.28, 9.72) |
| Range | (1.7*, 10.2) | | (2.0*, 7.4) | | (1.7*, 10.2) | |
| 6-month event-free rate (95% CI) | 0.464 | (0.252, 0.651) | 0.500 | (0.058, 0.845) | 0.471 | (0.275, 0.645) |
| 12-month event-free rate (95% CI) | 0.000 | (NE, NE) | 0.000 | (NE, NE) | 0.000 | (NE, NE) |
| 18-month event-free rate (95% CI) | 0.000 | (NE, NE) | 0.000 | (NE, NE) | 0.000 | (NE, NE) |
| 24-month event-free rate (95% CI) | 0.000 | (NE, NE) | 0.000 | (NE, NE) | 0.000 | (NE, NE) |

Note:
*= censored observation, NE = not estimable
ITT: BRCA or non BRCA subjects; BRCA: biallelic BRCA1 or BRCA2 or germline BRCA; non-BRCA; biallelic in ATM, FANCA, PALB2, CHEK2, BRIP1 or HDAC2

TABLE 13
Efficacy of niraparib in patients with mCRPC

| | Efficacy analysis (n = 223) | | | |
|---|---|---|---|---|
| Response, | BRCA | | Non-BRCA^{a} | |
| n (%) (95% CI) | (n = 142) | | (n = 81) | |
| ORR (measurable disease) | 26/76 (34.2) | | 5/47 (10.6) | |
| | (23.7-46.0) | | (3.5-23.1) | |
| CRR | 82/142 (57.7) | | 12/81 (14.8) | |
| | (49.2-66.0) | | (7.9-24.5) | |
| PSA₅₀ | 61/142 (43.0) | | 4/81 (4.9) | |
| | (34.7-51.5) | | (1.4-12.2) | |
| CTC Response (CTC0)^{a} | 31/131 | (23.7) | 6/71 | (8.5) |
| CTC conversion^{b} | 55/117 (47.0) | | 9/60 (15.0) | |
| | (37.7-56.5) | | (7.1-26.6) | |
| DOR, median (range), mo | 6.28 | (3.65-9.23) | 5.16 | (2.14-NE) |
| rPFS, median (95% CI), mo | 8.08 | (5.55-8.38) | 3.71 | (1.97-5.49) |
| OS, median (95% CI), mo | 13.01 | (11.04-14.29) | 9.63 | (8.05-13.44) |
| Time to radiographic progression, median (95% CI), mo | 8.08 | (5.75-8.97) | 3.78 | (2.00-5.55) |
| Time to SSE, median (95% CI), mo | 13.80 | (10.41, NE) | 10.35 | (8.18, NE) |

^{a}Defined per protocol and SAP as CTC = 0 per 7.5 mL blood at 8 weeks post-baseline in patients with baseline CTC >0.
^{b}Among patients with baseline CTC ≥5.
BRCA = breast cancer gene. CI = confidence interval. CRR = composite response rate. CTC = circulating tumor cells. CTC0 = CTC count >0 at baseline to 0/7.5 mL blood at week 8; CTC conversion, CTC count ≥5/7.5 mL blood at baseline to <5/7.5 mL blood at nadir. DOR = duration of objective response. NE = not estimable. ORR = objective response rate. OS = overall survival. PSA₅₀ = ≥50% decline in prostate-specific antigen. rPFS = radiographic progression-free survival.

### Safety Results

All subjects, except one, experienced at least 1 AE (99.7%) and 90% of subjects reported a related AE.
SAEs were reported by 46.4% of subjects.
AEs led to treatment discontinuation in 22.8% of subjects.
One subject reported a COVID-19-related AE, which was considered non serious. One death due to COVID-19 occurred, however this was outside the reporting period for treatment-emergent AEs.
Grade 3 or 4 AEs occurred in 75.1% of subjects. The most common were anemia (32.9%), thrombocytopenia (16.3%), and neutropenia (9.7%), fatigue (6.6%), and nausea (5.2%). The most common Grade 4 AE was thrombocytopenia in 23 (8.0%) subjects.
As of the data cutoff date, 26 (9%) of subjects had died within 30 days of the last dose of study drug. The majority of these deaths were due to progressive disease (17 [6%] subjects).
Deaths due to treatment-emergent AEs occurred in 16 (5.5%) subjects.
Adverse events (all grades) of clinical interest are: anemia (54.0%), thrombocytopenia (34.3%), neutropenia (19.4%), febrile neutropenia (1.0%) and neutropenic sepsis (0.3%).
The most frequent (≥2.5%) Grade 3 or 4 clinical chemistry values reported during the study were: increased GGT (5.0%): increased alkaline phosphatase and hyperkalemia (2.5% each); and hyponatremia (3.2%).
The most frequent (≥10% subjects) Grade 3 or 4 hematology values reported were: decreased lymphocyte count (18.4% subjects) and anemia (17.0% subjects).

### CONCLUSIONS

As of the clinical cutoff date of Jan. 26, 2021, median follow-up time for the primary efficacy population (BRCA measurable) was 10.0 months. Their median age was 66 years and 57.9% had an ECOG performance status of 1 and 9.2% had a 2 at study entry. Most subjects had significant burden of disease at baseline. 80% with bone disease and importantly 40% had visceral disease. Fifty-one subjects (67.1%) received 1 AR-targeted therapy of whom 37 had 1 taxane-based chemotherapy and 14 had 2 taxane-based chemotherapies. Twenty-five subjects (32.9%) had 2 AR-targeted therapies of whom 14 had 1 taxane-based chemotherapy and 11 subjects had 2 taxane-based chemotherapies. Most subjects received at least 1 taxane-based chemotherapy in the mCRPC setting.

The starting dose of niraparib monotherapy was 300 mg by mouth daily. The median treatment duration was estimated to be 6.47 months in the BRCA group (N=142) and 3.55 months in the non-BRCA group (N=81).

GALAHAD was the first study to evaluate niraparib in metastatic prostate cancer and utilized multiple assays to enrich for patients with DRD in 2 cohorts (BRCA and non-BRCA). Treatment with niraparib, in a more heavily pre-treated subject population with more advanced stage of disease relative to other trials of PARP inhibitors in mCRPC patients after at least second line treatment, resulted in an ORR of 34.2% in BRCA subjects. This finding is noteworthy in a patient population with few remaining therapeutic options and a high prevalence of visceral metastases (in nearly 40% of patients) at baseline. Radiographic progression-free survival and overall survival also tended to be longer in the BRCA cohort, with median rPFS being approximately double that in the non-BRCA cohort.

The niraparib safety profile in this heavily pre-treated prostate cancer population was manageable. For example, patients received blood transfusions or erythropoietin to treat anemia and maintain blood counts. The adverse events observed are consistent with the known safety profile of niraparib. Subjects in the primary efficacy population who achieved an objective response had a higher relative dose intensity and fewer interruptions compared to non-responders.

Taken together, these results extend the evidence for efficacy of PARP inhibitors in mCRPC patients with DRD and disease progression on prior treatments as well as further support the observation that precision medicine can offer meaningful benefit in this setting, especially in patients with BRCA1/2 mutations.

1. A method of improving the median overall survival (OS) in a male human with metastatic castration-resistant prostate cancer (mCRPC) with biallelic DNA-repair anomalies, wherein said biallelic DNA-repair anomalies are selected from: i) BRCA1, BRCA2, or a combination thereof, ii) ATM, FANCA, PALB2, CHEK2, BRIP1, HDAC2, or any combination thereof; or iii) any combination thereof; and wherein the male human has received prior taxane-based chemotherapy and androgen receptor (AR)-targeted therapy, the method comprising administering to such male human a once-daily oral dosing of 300 mg niraparib and the OS is least about 12 months with a 95% confidence interval (95% CI).

2. The method of improving OS of claim 1, wherein the male human has BRCA DNA-repair anomalies and the median OS is about 13 months (95% CI).

3. The method of improving OS of claim 1, wherein the male human has DNA-repair anomalies selected from ATM, FANCA, PALB2, CHEK2, BRIP1, HDAC2, or any combination thereof, and the median OS is about 10 months (95% CI).

4. The method of improving OS of claim 1, wherein there is a radiographic progression-free survival (rPFS) of about 5.6 months (95% CI).

5. The method of improving OS of claim 4, wherein the male human has BRCA DNA-repair anomalies and the rPFS is about 8.1 months (95% CI).

6. The method of improving OS of claim 1, wherein the male human has BRCA DNA-repair anomalies and the improved efficacy is an objective response rate (ORR) of about 34.2% (95% CI).

7. The method of improving OS of claim 1, wherein there is a median duration of objective response of about 5.55 months (95% CI).

8. The method of improving OS of claim 1, wherein there is median time to radiographic progression of about 5.8 months (95% CI).

9. The method of improving OS of claim 8, wherein the male human has BRCA DNA-repair anomalies and the median time to radiographic progression is about 8.08 months (95% CI).

10. The method of improving OS of claim 1, wherein there is a median time to PSA progression of about 4.6 months (95% CI).

11. The method of improving OS of claim 1, wherein there is a median time to symptomatic skeletal event of about 13 months (95% CI).

12. The method of improving OS of claim 1, wherein there is a circulating tumor cells (CTC) response rate of about 18% (95% CI).

13. The method of improving OS of claim 12, wherein the male human has BRCA DNA-repair anomalies and the CTC response rate is about 24% (95% CI).

14. The method of improving OS of claim 12, wherein the male human has DNA-repair anomalies selected from ATM, FANCA, PALB2, CHEK2, BRIP1, HDAC2, or any combination thereof, and the CTC response rate is about 9% (95% CI).

15. The method of improving OS of claim 1, wherein the taxane-based chemotherapy is selected from docetaxel, paclitaxel, or cabazitaxel.

16. The method of improving OS of claim 1, wherein the AR-targeted therapy is selected from the group consisting of surgical castration (orchiectomy); leuprorelin or leuprolide, goserelin, triptorelin, histrelin, nafarelin, gonadorelin, buserelin, relugolix; abiraterone acetate, ketoconazole, flutamide, nilutamide, bicalutamide, enzalutamide, apalutamide, and darolutamide.

17. The method of improving OS of claim 1, wherein niraparib is in the salt form selected from the group consisting essentially of tosylate monohydrate, sulfate, benzenesulfate, fumarate, succinate, camphorate, mandelate, camsylate, lauryl sulfate, and a mixture of tosylate monohydrate and lauryl sulfate.
